(19) United States
(12) Patent Application Publication
(10) Pub. No.: US 2025/0073473 A1
(43) Pub. Date: Mar. 6, 2025

(54) TRANSVASCULAR BRAIN STIMULATION

(71) Applicant: Synchron Australia Pty Limited, Dover, DE (US)

(72) Inventors: Michael HAINES, Ormond (AU); Stephen Michael RONAYNE, Parkville (AU); Gil Simon RIND, Northcote (AU); Thomas James OXLEY, Dover, DE (US)

(73) Assignee: Synchron Australia Pty Limited, Dover, DE (US)

(21) Appl. No.: 18/823,453

(22) Filed: Sep. 3, 2024

Related U.S. Application Data
(60) Provisional application No. 63/580,304, filed on Sep. 1, 2023.
(60) Provisional application No. 63/596,220, filed on Nov. 3, 2023.
(60) Provisional application No. 63/552,617, filed on Feb. 12, 2024.

(51) Int. Cl. A61N 1/372; A61N 1/05; A61N 1/36

(52) U.S. Cl. CPC A61N 1/372; A61N 1/0534; A61N 1/3605

(57) ABSTRACT

Methods and devices for transvascular placement of electrodes in on a surface of a brain or in deep brain structures for the purpose of neuromodulation.

### CROSS-REFERENCE TO RELATED APPLICATIONS

This application is a non-provisional application of U.S. provisional application No. 63/580,304 filed Sep. 1, 2023, 63/596,220 filed Nov. 3, 2023, and 63/552,617 filed Feb. 12, 2024, the entirety of each of which are incorporated by reference.

### BACKGROUND

Presently, conventional approaches exist that attempt to access regions of the brain for stimulation of neural tissue or detecting neural signals. Such approaches that are generally known include deep brain stimulation (“DBS”), which involves implanting electrodes within certain areas of a brain where the electrodes produce electrical impulses in an attempt to stimulate or regulate brain activity for a therapeutic or other purpose, as well as electrocorticography (“ECoG”), which enables neuromonitoring of brain regions for a diagnostic purpose.

Typically, implantation of such neural devices involves creating burr holes in the skull to implant electrodes and surgery to implant a controller or pacemaker-like device that is electrically coupled to the electrodes to control the stimulation or to sense neural signals. This device can be positioned under the skin in the chest. The amount of stimulation in deep brain stimulation can be controlled by the controller or pacemaker-like device where a wire/lead connects the controller device to electrodes positioned in the brain.

DBS can be used to treat a number of neurological conditions, such as tremors, Parkinson's disease, dystonia, epilepsy, Tourette syndrome, chronic pain, and obsessive-compulsive disorder. In addition, DBS has the potential for treatment of major depression, stroke recovery, addiction, and dementia. Moreover, implanting electrodes in neural tissue can influence the efficacy of stimulating and/or recording neural tissue (e.g., using brain-computer interfaces), such as decoding thoughts from neural signals.

The positioning of electrodes on the brain or into neural tissue can present risks, especially when using a transcranial approach. FIG. 1 illustrates a conventional transcranial approach of accessing regions of the brain 12 of an individual 10 with a brain stimulation/monitoring device 20, usually including an electrode carrier 22, having a plurality of electrodes 24 that are implanted within a target region 30 of the brain 12. As shown, the implantation requires surgical penetration, e.g., a craniotomy, of the skull 14 such that the electrodes 24 are directed towards an area of interest 30. In addition, the device 20 includes a lead 16 that couples the electrodes 24 to a controller/transceiver/generator 26. The lead 16 and controller 26 can be surgically implanted within the individual 10 or positioned on an exterior surface of the individual 10.

There are a number of risks associated with the general surgery required to surgically implant the device 20 in conventional DBS procedures. Furthermore, there are risks in the process of the DBS procedure itself, given that conventional procedures require an approximation or non-invasive attempt to locate the region of interest 30. Then, the physician must attempt to physically position the electrodes 22 of the device 20 in or near the area of interest 30 so that the desired effect can be achieved. In certain cases, the positioning of the electrodes 20 can be a trial-and-error approach requiring multiple surgical attempts and multiple surgical insertion sites. Regardless of the number of attempts, the act of inserting the device 20 to position the electrodes 22 in the area of interest 30 creates collateral damage to brain tissue located in the path between the area of interest and the insertion point in the cranium.

Currently, the surgical risks involved in such procedures can include bleeding in the brain, stroke, infection, collateral damage to brain tissue, collateral damage to vascular structures in the brain, temporary pain, and inflammation at the surgical site.

However, the conventional approaches intended to access the many subnetworks of the brain are deficient such that the conventional approaches are unable to maximize the benefit of accessing and directly communicating/stimulating these subnetworks. For example, in the case of using a brain stimulation device 20 to treat Parkinson's disease, an electrode 24 or electrode carrier 22 must be positioned through a significant amount of brain structures to ensure the electrode 24 is positioned at or near a target site 30. Once positioned, either the lead 16 or the electrode carrier 22 comes out through the skull 14 under skin and then is positioned to reach the controller 26, which is typically positioned on or in the chest.

Neurovascular electrophysiology and therapeutic devices are limited in their positioning over or within the cortex by the highly variable physical presence and pathway that veins take. Therefore, to gain access to wider regions of functionally rich brain regions for recording and stimulation purposes, the ability to deploy recording and stimulation arrays without the spatial limitations of the vascular network will prove highly valuable.

There remains a need for implantation of electrodes and/or neural sensing/stimulation devices while minimizing collateral damage to tissue from the procedure. There especially remains a need for a transvascular approach to create a location or space within the dura matter so that a vascular approach can deliver electrodes or other devices to the space. There also remains a need for deploying electrode steering devices to locations adjacent to or in brain tissue and closing vessel punctures post-delivery.

It is noted that the devices discussed herein can allow transvascular placement to position electrodes in deep brain structures for the purpose of neuromodulation, including movement disorders, epilepsy, and depression. The electrodes can reside in a deep brain region in an intraparenchymal location with a penetrating electrode array. Alternatively, the electrodes can be surface electrodes. These devices are able to sense and stimulate the brain region to reduce a particular symptom (e.g., tremor in Parkinson's or seizures in epilepsy. The devices can be open-loop or closed-loop. In addition, the electrode devices can perform intracranial electroencephalography such as ECoG, for neuromonitoring of brain regions.

The following U.S. patents describe the use of the venous network to access brain tissue in order to form a shut to relieve cranial pressure: U.S. Pat. No. 9,387,311 issued on Jul. 12, 2016, U.S. Pat. No. 9,545,505 issued on Jan. 17, 2017, U.S. Pat. No. 9,662,479 issued on May 30, 2017, U.S. Pat. No. 9,669,195 issued on Jun. 6, 2017, U.S. Pat. No. 10,272,230 issued on Apr. 30, 2019, U.S. Pat. No. 9,724,501 issued on Aug. 8, 2017, U.S. Pat. No. 10,279,154 issued on May 7, 2019, U.S. Pat. No. 10,058,686 issued on Aug. 28, 2018, U.S. Pat. No. 10,758,718 issued on Sep. 1, 2020, U.S. Pat. No. 10,765,846 issued on Sep. 8, 2020, U.S. Pat. No. 10,307,576 issued on Jun. 4, 2019, U.S. Pat. No. 10,307,577 issued on Jun. 4, 2019, U.S. Pat. No. 11,013,900 issued on May 25, 2021, U.S. Pat. No. 11,633,578 issued on Apr. 25, 2023, the entirety of which is incorporated by reference. The present disclosure can incorporate such access and provides novel methods, devices, and systems for locating, directing, and/or implanting neural sensing/stimulation devices within deep brain tissue.

### SUMMARY OF THE INVENTION

The present disclosure includes methods, devices and systems that enable deposition of a electrodes and other recording devices in information rich areas of the brain or the deposition of closed loop feedback implantable brain stimulator via the venous system of the brain.

An example of such a system can include multiple elements that permit venous access via a catheter that delivers a guide catheter from jugular vein and punctures into inferior petrosal sinus.

Variations of the present disclosure include systems for accessing a target region of a brain from a vessel. For example, such a system can include a catheter body having a distal region; a navigation device slidably advanceable through the catheter body to the distal region, the navigation device including a distal portion that is configured to be steerable independently of the catheter body and an expandable member at the distal portion, where the expandable member is configured to anchor the distal portion exterior to the vessel; a guidewire configured to extend through a working lumen of the navigation device; and an electrode carrier configured to be advanced through the working lumen of the navigation device and through the expandable member such that the electrode carrier can be advance in a straight line from an opening in the expandable member to the target region of the brain.

Variations of the present disclosure can also include a first expandable structure located at the distal region of the catheter body and configured to bias the catheter body against a wall of the vessel.

The systems described herein can include a catheter body that includes a passage exiting a side opening in a sidewall at the distal region, wherein the passage is configured such that advancement of the navigation device therethrough causes the navigation device to exit the catheter body at the side opening.

Variations of the present disclosure can include systems that further include a bone penetrating structure configured for sliding through the catheter body.

The electrode carrier described herein can include a linear electrode array, an electrode array having a planar electrode region configured to have a delivery profile and expandable to a planar profile when advanced out of the navigation device, an array where the planar electrode region includes a foldable structure such that expansion of the planar electrode region from the delivery profile to the planar profile includes unfolding the foldable structure; and/or an array with a planar electrode region that includes an expandable structure such that expansion of the planar electrode region from the delivery profile to the planar profile includes expanding the expandable structure to expose one or more electrodes.

Variations of the present disclosure include a system having a grommet structure configured for placement within an opening in a wall of the vessel, where the grommet structure allows passage of the catheter body or navigation device therethrough.

The present disclosure can include a system having a stent structure having at least one opening in a side of the stent structure for passage of the catheter body or navigation device therethrough when positioned in the vessel.

The stents disclosed herein can include a stent body expandable from a deployment configuration to an expanded configuration; a port extending from a side of the stent body, the port having a passage and having a sharp edge on a free end of the port opposite to the stent body; a polymer covering the port and the sharp edge, wherein the polymer is configured to dissolve or degrade over a period of time, wherein when deployed in a vessel the stent body biases the polymer covering the sharp edge against a wall of the vessel, wherein after the polymer dissolves or degrades, the stent body urges the sharp edge of the port into the wall of the vessel such that the wall of the vessel adheres to a portion of the port to secure the port in place.

The present disclosure also includes methods of transvascular access of a region of a brain. For example, such methods can include advancing a catheter into a vessel; anchoring the catheter within the vessel; passing the catheter through a vessel opening in a wall of the vessel and adjacent to brain tissue; deploying a navigation device from the catheter to an exterior of the vessel; expanding an expandable structure located at a distal portion of the catheter, where the expandable structure anchors to the exterior of the vessel; steering the expandable structure to align a travel path from an opening of the expandable structure to a target region; and advancing an electrode carrier from the opening of the expandable structure along the travel path and to the target region.

The methods described herein can include an electrode carrier that is advanced over a surface of the brain. Alternatively, or in combination, the methods can include advancing the electrode carrier from the opening of the expandable structure along the travel path and to the target region includes advancing the electrode carrier through a tissue of the brain.

Variations of the present disclosure include a method, further including expanding the electrode carrier in a planar a planar direction over the target region.

Variations of the present disclosure include a method, wherein the electrode carrier is configured to form a two dimensional array when expanded.

Variations of the present disclosure include a method, wherein expanding the electrode carrier in the planar direction includes unfolding the electrode carrier from a folded state.

The methods described herein can include a catheter with a biasing portion of the catheter that urges the catheter against a wall of the vessel.

Additional variations of the present disclosure include methods for transvascular access of a region of a brain. Such methods can include advancing a catheter into a vessel; anchoring the catheter within the vessel; passing the catheter through a vessel opening in a wall of the vessel and adjacent to brain tissue; deploying a navigation device from the catheter to an exterior of the vessel; expanding an expandable structure located at a distal portion of the catheter, where the expandable structure anchors to the exterior of the vessel; steering the expandable structure to align a travel path from an opening of the expandable structure to a target region; and advancing an electrode carrier from the opening of the expandable structure along the travel path and to the target region.

Additional variations of the present disclosure can include include advancing a catheter into a vessel where a distal portion of the catheter includes at least one lumen terminating in a side opening in a sidewall of the catheter; anchoring the catheter within the vessel advancing a puncture catheter through the side opening of the catheter and through a wall of the vessel to create a vessel opening in the wall of the vessel; deploying an intermediate catheter over the puncture catheter into the vessel opening to and adjacent brain tissue; expanding one or more anchor members on the intermediate catheter to secure the intermediate catheter in place while extending through the vessel opening; removing the puncture catheter; an electrode carrier through the intermediate catheter and towards the region of the brain; removing the intermediate catheter; delivering a substance from the catheter to seal a portion of the electrode carrier within the vessel opening; and removing the catheter such that the electrode carrier is positioned transvascularly within the brain, expanding an expandable structure located at a distal portion of the catheter, where the expandable structure anchors to an exterior of the vessel; steering the expandable structure to align a travel path from an opening of the expandable structure to a target region; and advancing an electrode carrier from the opening of the expandable structure along the travel path and to the target region.

In another variation, the methods can include delivering a needle from the guide catheter that punctures the wall of the venous sinus (e.g., inferior petrosal sinus) and skull to enter the brain tissue and then delivering a steerable navigational device from the exterior of the vessel through a wall of the skull and into the brain. The device can include one or more anchors that anchor the catheter into position to permit targeted deployment of an electrode lead into the brain.

In another variation, the method can include manipulating the navigational device such that it can be repositioned in a 3 dimensional space to precisely target a straight-line trajectory for the entry of the lead into the brain. The position of the anchor would manipulate the position of the catheter in relation to the entry position with relation to the brain, including:

The navigation device can include any number of sensors or markers that allow for non-invasive imaging to confirm positioning of the electrodes. Alternatively, or in combination, confirming the position of the anchor in 3D space can occur with a 2-way communication of an external stereotactic navigation system.

In another variation, the system can use an external magnetic system for manipulation of the navigation device.

Targets include all known deep brain stimulation targets. One example is the subthalamic nucleus to treat tremor associated with Parkinson's disease (which can be 20 mm away from the inferior petrosal sinus).

The present disclosure can be used in addition to the devices disclosed in the following patents/publications or in combination with aspects and features of the related disclosure of these patents, publications, and applications: U.S. Pat. No. 10,575,783 issued on Mar. 3, 2020, U.S. Pat. No. 10,485,968 issued on Nov. 26, 2019, U.S. Pat. No. 10,729,530 issued on Aug. 4, 2020, US20190336748 published on Nov. 7, 2019, US20200016396 published on Jan. 16, 2020, US20220253024 published on Aug. 11, 2022, U.S. Pat. No. 11,550,391 issued on Jan. 10, 2023, U.S. Pat. No. 11,672,986 issued on Jun. 13, 2023, US20220369994 published on Nov. 24, 2022, and U.S. Pat. No. 11,630,517 issued on Apr. 18, 2023, and U.S. application Ser. No. 18/792,965 filed on Aug. 2, 2024. The entirety of each of these is incorporated by reference.

### BRIEF DESCRIPTION OF THE DRAWINGS

FIG. 1 illustrates a conventional transcranial approach of accessing regions of the brain of an individual with a brain stimulation/monitoring device.

FIG. 2A illustrates an access device advanced into a jugular vein to permit navigation of a catheter within an inferior petrosal sinus.

FIG. 2B shows a directing structure advanced from a catheter.

FIG. 3A illustrates a variation of a navigation device with a second anchor and illustrates an intended travel path from the navigation device.

FIG. 3B illustrates the navigation device of FIG. 3A being manipulated to alter the intended travel path to coincide with a region of interest.

FIG. 4A shows a variation of a navigation device to position an electrode carrier along alignment path to place electrodes within or near a region of interest.

FIG. 4B illustrates a state where the catheter, navigation device, anchor, and other delivery elements are removed from the site to leave the electrodes positioned within the region of interest.

FIGS. 5A to 5F shows another variation of a transvascular approach to position an electrode carrier within or near a region of interest.

FIG. 5G shows a partial cross-sectional view of an electrode carrier having a magnetic stylet extending within the carrier.

FIG. 6 illustrates implanted electrodes positioned within a region of interest in the brain and where a lead or electrode carrier extends partially through the vasculature towards a controller.

FIGS. 7A to 7F illustrate examples of a process of accessing the subarachnoid cavity or other deep brain region.

FIGS. 8A to 8C show a stent structure having a port with a sharp distal edge transitioning to a medical textile brim section on the proximal side.

FIGS. 9A and 9B illustrate a self-expanding or balloon expanded stent frame with large cell size with a region of thin, soft, self-healing, polymer material between cell(s).

FIGS. 10A to 10C illustrate an optional design of a recording array for deployment through the vessel wall.

FIGS. 11A to 11C illustrate a folding array that interchanges between two deployment states.

FIGS. 12A to 12B illustrate an expandable electrode carrier extending out of a superior sagittal sinus for positioning over brain tissue.

FIG. 12C illustrates a guide catheter within a vessel, where the catheter includes expanding features to secure a side port against a wall of the vessel.

FIGS. 13A to 13D illustrate additional variations of devices that form planar electrode carriers.

FIGS. 14A and 14B illustrate perspective and side views respectively of an example of an expanding vessel puncture port or grommet.

### DETAILED DESCRIPTION

The present methods and devices relate electrodes that directly accessing, monitoring, and/or communicating with specific regions of the brain via a vascular approach for the purpose of using the direct access to minimize damage to adjacent tissues within the brain and anatomy.

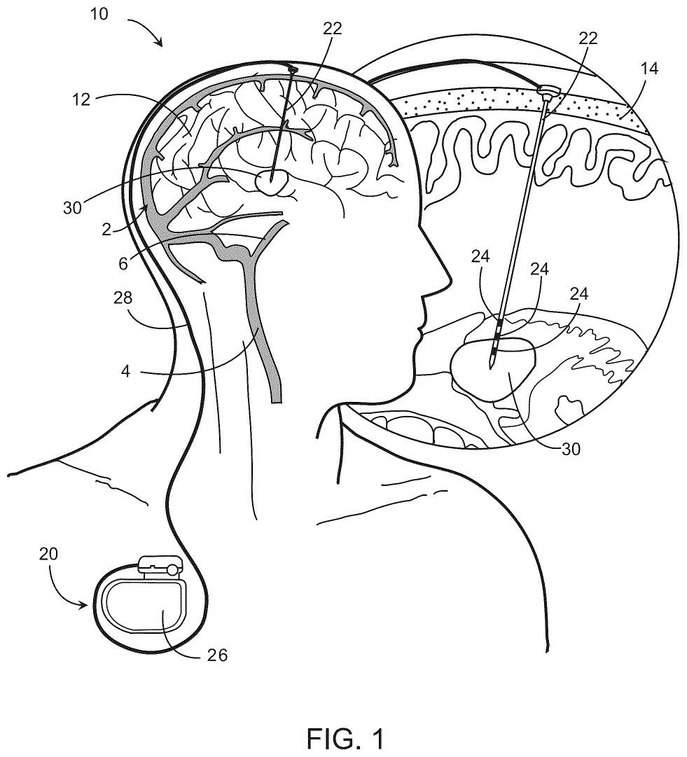

FIG. 1 also illustrates a vascular network 2 of the brain extending from the jugular vein 4. The present disclosure uses the venous network 2 of the brain 12 to access target regions of interest for DBS. While the examples below discuss accessing the inferior petrosal sinus (IPS) 6 that branches from the jugular vein 4, any vessel located within the brain 12 can be used for access of neural tissue.

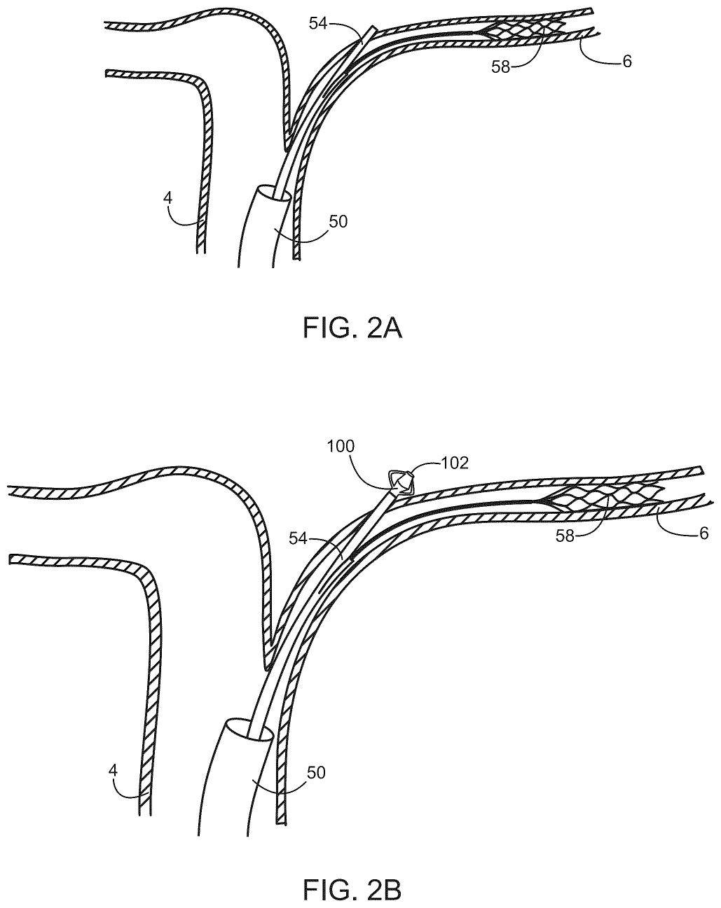

FIG. 2A illustrates an access device 50 advanced into the jugular vein 4 to permit navigation of a catheter 54 within a vessel 6, in this case the vessel comprises the IPS. As shown, the catheter 54 can include an anchor or stent 58 to secure the catheter 54 at the desired location in the vessel. The catheter 54 can include any number of mechanisms to puncture the wall of the IPS 6 as well as any tissue (e.g., the skull, etc.) to provide an access to brain tissue. It is noted that any venous and/or arterial approach is within the scope of this disclosure. Moreover, the procedure can include any of the patents discussed above that use the venous network to access brain tissue in order to form a shut to relieve cranial pressure.

FIG. 2B shows a directing structure 100 advanced from the catheter 50. As discussed below, the directing structure 100 can function to anchor the catheter 54 as well as articulate/rotate to provide navigational capabilities to direct an electrode from an opening 102 in the directing structure 100 to a region of interest within brain tissue. The navigational capabilities can include three-dimensional navigation such as articulation and/or rotation. The directing structure 100 can include any number of steering mechanisms commonly used for articulation of devices, including magnetic positioners, motors, shape memory alloys, pull-wires, etc.

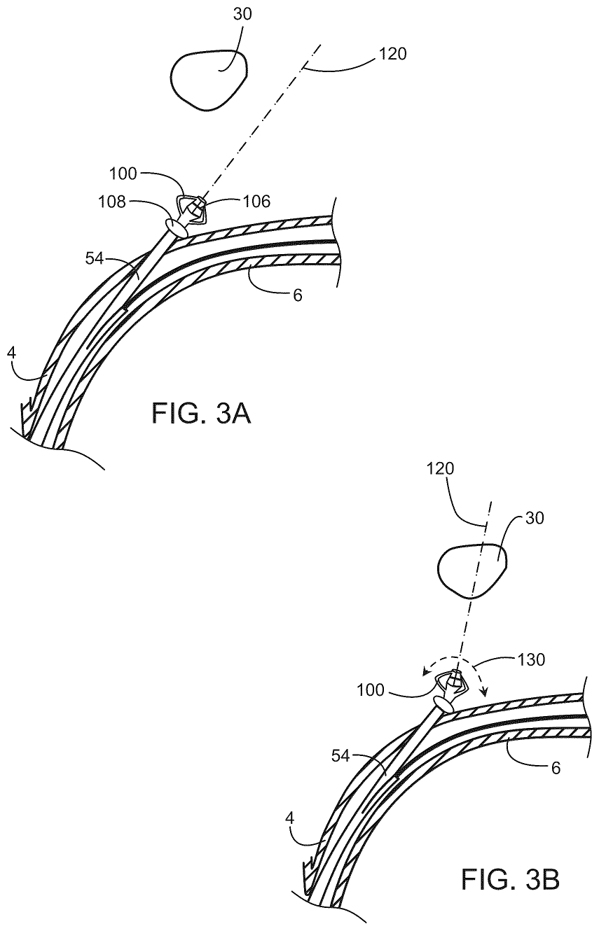

FIG. 3A illustrates a variation of a navigation device 100 similar to that shown in FIG. 2B. The navigation device 100 of FIG. 3A optionally includes a second anchor 108 to allow spacing and/or 3-dimensional movement of the navigation device 100. FIG. 3A also illustrates an exemplary target region 30, similar to that shown in FIGS. 2A and 2B. In operation, navigation device 100 (or another part of the components) will include one or more sensors, beacons, or radiopaque markers 106 that allow a user to identify a potential travel path 120 of an electrode or similar structure that would exit the navigation device 106 in the present orientation. As noted above, the navigation device 100 will include steering features such that it can navigate in a three dimensional plane, articulate, rotate, or otherwise be repositioned (as shown by arrows 130) such that the potential travel path 120 aligns with a desired region 30 as shown in FIG. 3B. For example, the navigation device can be steered via one or more steering wires. Alternatively, or in combination, the navigation device can be steered using a magnetic field as discussed below. In some variations of the system, a two-way communication system ensures the navigation device 100 moves in three dimensional space to a desired orientation. Such a system can include external navigation imaging (e.g., CT scan data, fluoroscopic imaging, ultrasound imaging, stereotactic surgical navigation systems, etc.)

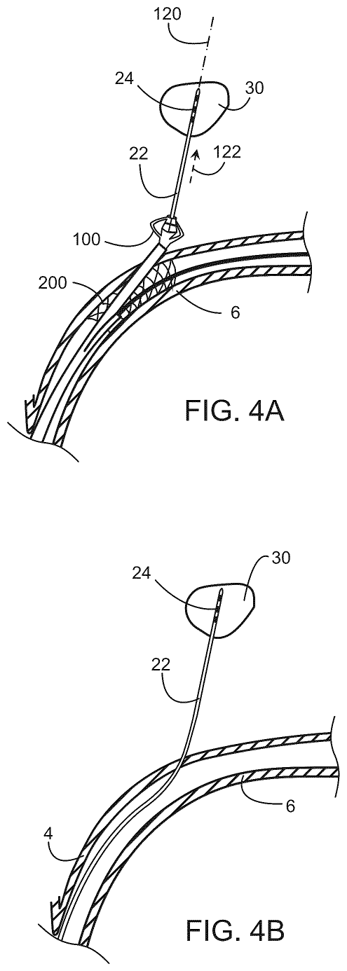

FIG. 4A shows the device navigation device 100 in position as an electrode carrier 22 advances in direction 122 along alignment path 120 until electrodes 24 are positioned within or near a region of interest 30. While not shown, the device 100 can advance a needle or other cannula for positioning the electrodes 24 within the region of interest 30. FIG. 4A also illustrates a variation where a stent 180 is used within the vessel 6 to assist in transvascular access. Examples of such stents are discussed below.

FIG. 4B illustrates the state where the catheter, navigation device, anchor, and other delivery elements are removed from the site to leave the electrodes 24 positioned within the region of interest 30 and the electrode carrier 22 or lead extending back into the IPF 6 wall and back through the jugular vein.

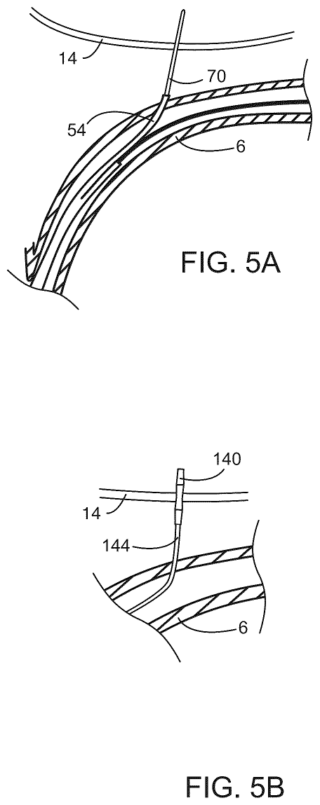

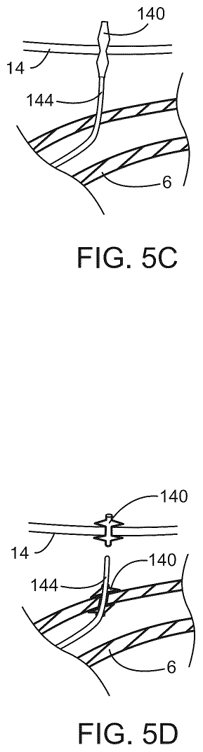

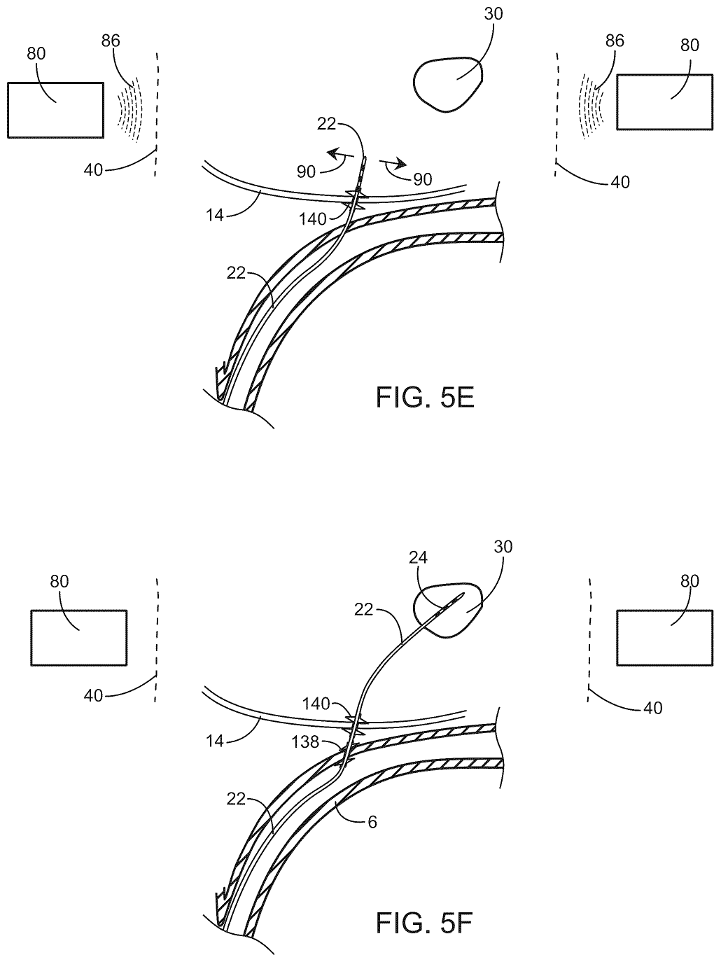

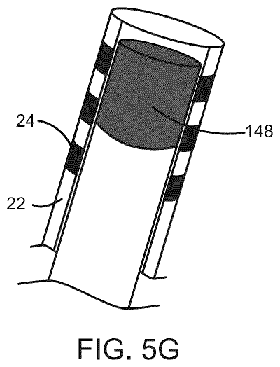

FIG. 5A shows a catheter 54 or similar device within the IPS 6. As shown, the catheter 54, as noted above in FIG. 2A, one or more anchors can be used to secure the catheter 54 at the desired location in the vessel. The catheter 54 can include any number of mechanisms to puncture the wall of the IPS 6 as well as any tissue (e.g., the skull, etc.) to provide an access to brain tissue. As shown a needle 70 can be used to penetrate the IPS 6 as well as the skull 14. Again, the approach can be a venous and/or arterial approach. FIG. 5B illustrates a deployment stylet 144 positioning a fixation device 140 within the skull 14. In alternate variations, the same catheter 54 can be used to position the fixation device 140.

FIG. 5C illustrates actuation of the fixation device 140 such that a portion of the fixation device 140 deploys about the skull 14. The fixation device 140 can be self-expanding, or expand through actuation (e.g., mechanical, electrical, temperature, etc.). Ultimately, as shown in FIG. 5D, the fixation device 140 is in a deployed configuration about the skull 14 to provide a pathway therethrough. It is also noted that in additional variations, a fixation device 138 can be positioned about the vessel wall 6.

FIG. 5E illustrates the fixation device 14 with an electrode carrier 22 extending therethrough. In this variation, the electrode carrier can be navigated using one or more magnets 80. In one example, the magnets 80 are positioned outside the body, as denoted by lines 40), such that one or more magnetic fields 86 can alter a trajectory of the electrode carrier as denoted by arrows 90 as the carrier advances distally towards a region of interest 30. Ultimately, as shown in FIG. 5F, the electrodes 24 on the electrode carrier 22 are positioned within or near to the region of interest. FIG. 5F also shows a variation in which an anchor 138 is positioned within the vessel 6 such that there is anchoring 138, 140 both within and outside of the vessel 6.

FIG. 5G shows a partial cross-sectional view of an electrode carrier 22 having a magnetic stylet 148 extending within the carrier 22. The magnetic stylet can be removable from the carrier 22 once the electrodes 24 are positioned. The removable stylet 148 permits the carrier 22 to remain implanted within the body without a magnetic component. The catheters and navigation devices described herein can also have similar configurations to permit navigation or movement through the use of a magnetic field.

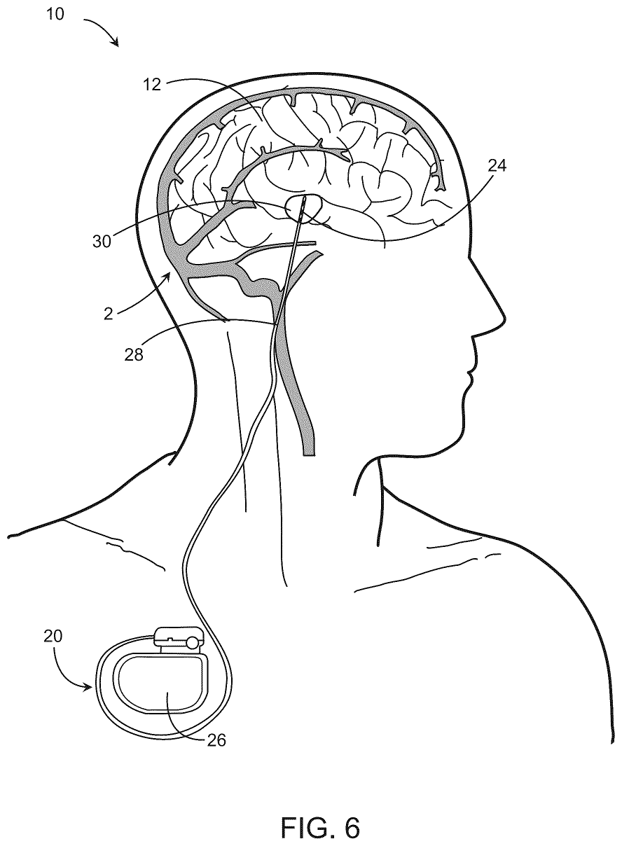

FIG. 6 illustrates the implanted electrodes 24 positioned within a region of interest 30 in the brain and where a lead or electrode carrier 24 extends partially through the vasculature 2 towards the controller 26 such that the stimulation/monitoring device 20 is implanted within the individual while minimizing collateral damage from conventional DBS procedures.

Neurovascular electrophysiology and therapeutic devices are limited in their positioning over or within the cortex by the highly variable physical presence and pathway that veins take. To gain access to wider regions of functionally rich brain regions for recording and stimulation purposes, the ability to deploy recording and stimulation arrays without the spatial limitations of the vascular network will prove highly valuable. The ability to safely deliver devices to the same brain regions, without the need for a craniotomy is therefore advantageous for patient safety.

The present disclosure also describes methods for the delivery of EP device, via the vascular system, to the subarachnoid cavity adjacent to cortical areas of interest via transvascular approaches. First, there is a need to access the subarachnoid cavity. There is also a need for a recording array for placement within the subarachnoid cavity. In additional variations, the array can be positioned within other deep brain regions as well.

The following methodologies are examples of accessing the subarachnoid cavity: access vasculature through standard neurointerventional technique via neck, femoral, or radial puncture; navigate to target puncture site using available neurointerventional imaging modalities, such as C-Arm fluoroscopy; anchor guide within vasculature at target puncture site; puncture through the vessel wall into the subdural space; feed a deployment catheter or device through puncture to subdural space and navigate to deployment site; deploy device, and remove relevant delivery tools.

Although the below methods focus on maintaining vascular blood flow, it is possible to navigate through a sacrificial vessel such as the middle meningeal artery, block the vessel, and follow similar steps of access to the subdural space as an alternative.

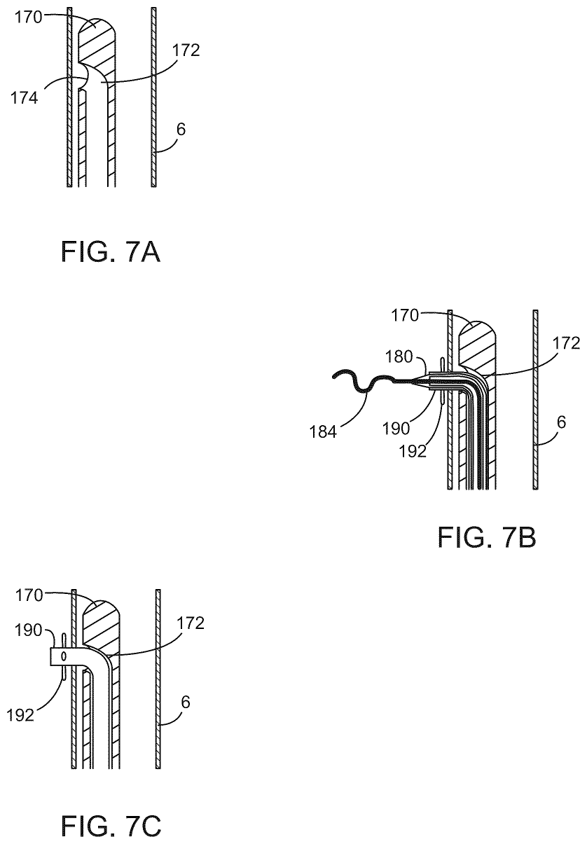

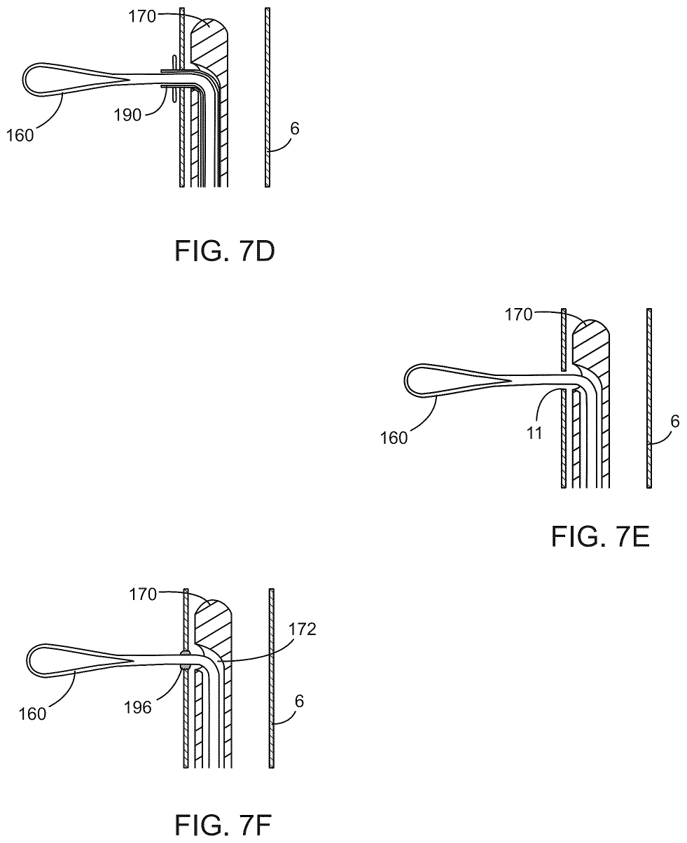

FIGS. 7A to 7F illustrate examples of a process of accessing the subarachnoid cavity or other brain region for delivery of an electrode array through a vessel 6. FIG. 7A shows a directional catheter 170 having a directional lumen 172 that terminates in an opening 174 in a sidewall of the catheter 170. The directional lumen 172 can have any number of deflecting surfaces located therein. Though not shown, variations of a directional catheter 170 can include one more expanding features to secure the catheter within blood vessel, where such features are disclosed herein, including but not limited to expanding struts/balloon/stent frame, etc.

FIG. 7B shows the directional catheter 170 having an optional guidewire 184 extending through a puncture catheter 180 that extends through the directional lumen 172 and through the opening 174 in the sidewall to puncture a wall of the vessel 6. The puncture catheter 180 can comprise any flexible structure (e.g., laser cut SS, shape memory alloy, polymeric, etc.). In one variation, the puncture catheter 180 comprises a micro, hypo-tube catheter, which can be a stainless steel hypo-tube or a hypo-tube comprised of other suitable materials, having a tapered, sharp distal tip for puncturing vascular tissue. The catheter 180 can also include a flexible section achieved via laser cut flexibility features, and an internal lumen through which the guidewire 184 can be delivered.

In the example shown, the puncture catheter 180 is advanced through an intermediate catheter 190. Once the puncture catheter 180 accesses the extra vascular space, a portion of the intermediate catheter 190 is positioned exterior to the vessel 6 wall and one or more expanding structures 192 are used to secure the intermediate catheter 190 in place. FIG. 7B shows the expanding structures as ribs 192 or a malecot. However, any anchoring structure (e.g., balloons, tines, etc.) can be used.

In some variations, a needle wire is used in place of a guidewire. This allows the operator to advance the piercing catheter 180 into the vessel wall, carefully penetrating through and into the extravascular space using the needle in a similar manner as used in cardiac procedures.

FIG. 7C shows the intermediate catheter 190 after removal of the puncture catheter and guidewire such that the intermediate catheter 190 provides an access path to the extra vascular space. The intermediate catheter 190 can include an outer polymer jacket that deploys the intermediate fixation feature 192 by sliding on the intermediate catheter distally, causing the structure 192 to prolapse outward and expand. This expansion can ensure catheter stability for the remainder of the procedure and mitigate the risk that the catheter is accidentally retracted across the puncture point. The fixation fixture may have a flexible polymer skin to form a temporary seal around the puncture site if leaking is present due to a pressure differential between CSF and venous system.

FIG. 7D shows advancement of an electrode carrier 160 into extravascular space through the intermediate catheter 190.

The Once the electrode carrier 160 is deployed in the transvascular space through an opening 11 in the vessel, the intermediate catheter can be withdrawn by disengaging the fixation feature for removal from the directional catheter 170 as shown in FIG. 7E.

FIG. 7F shows a variation where the operator can inject a polymer material 196 such as an adhesive (e.g., a cyanoacrylate) through the directional catheter lumen 172, directly onto the puncture site to enhance puncture closure and to secure the transvascular devices position. After a sufficient time, t directional catheter 170 is removed from the site leaving the electrode carrier 160 securely deployed in the extravascular space.

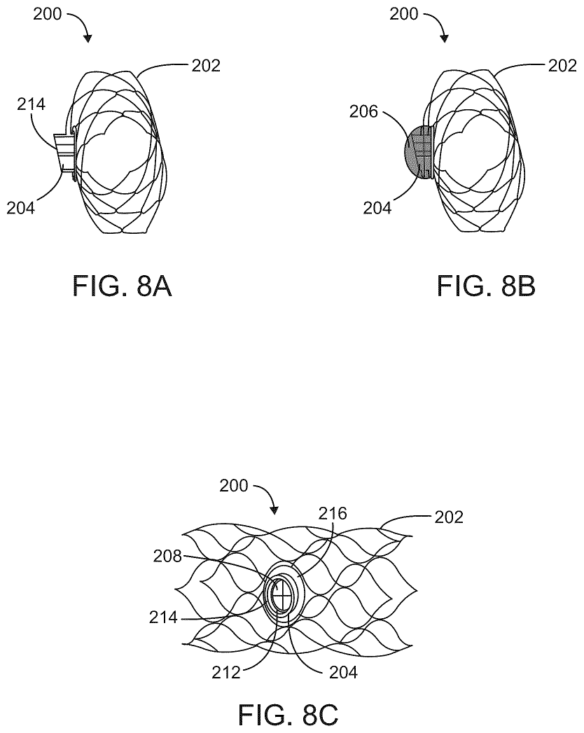

FIGS. 8A to 8C show an example of a stent structure 200 having a stent body 202 that can be positioned within a vessel. The stent body 202 can include a port 204 having a lumen 212. In one variation, an edge 214 of the port 204 that is opposite to the stent body 202 comprises a sharp edge transitioning to a medical textile brim section 216 on the proximal side or stent side. The port 204 includes a lumen 212 and can optionally have a valve or seal component 208 may be included to mitigate transgression of fluid across the open port lumen. In one variation, as shown in FIG. 8B, the port 204 includes a bio-dissolvable or degradable material that encases the port 204 or over the sharp edge 214. This material can be configured for slow or fast dissolving (e.g., (e.g., order of ˜2 weeks) within the blood stream to allow a controlled exposure rate of the sharp leading edge 214 of the port 204 to a wall of the vessel. The stent body 202 can be designed to provide constant outward radial force on the port 204, allowing controlled penetration of the port through the vascular tissue. A guide tube may be connected to the puncture lumen, trailing through the vasculature to the puncture site to aid later navigation and deployment through the puncture. Once the operator gains access to the target vessel with a guide catheter, the operator delivers the port stent device through the guide catheter, carefully positioning the port feature at the target portion of the vein where extra vascular access is required.

The operator deploys the stent port (self-expanding or balloon expanded), then removes the guide catheter and ends the procedure.

Either immediately, or after a period (e.g., 2 weeks) CT or similar imaging protocols can be used in a follow-up procedure to deploy a transvascular device through the port and into the extra vascular space. If a guide tube is present, the transvascular device is fed through the tube to the port. If a guide tube is not present, standard neurointerventional techniques would be used to navigate to the port.

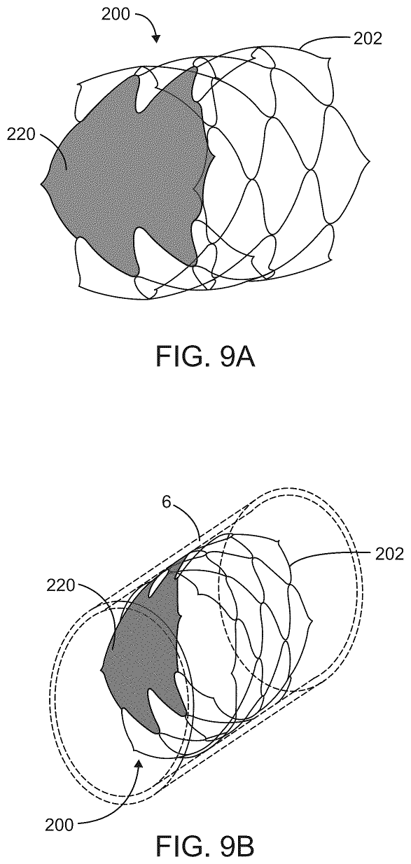

FIGS. 9A and 9B show an example of a self-expanding or balloon expanded stent 200 where a stent body 202 includes a frame with large cell size with a region of thin, soft, self-healing, polymer material 220 over an opening in the stent body 202. The polymer material 220 can be a mesh or single layer polymer sheet that is designed to be pulled taught by the expanding stent and produce a parallel plane arrangement with a wall of the vessel 6. The polymer 220 can be configured to provide mechanical support for the puncture and deployment system and provides an improved barrier seal from blood/CSF flow across the puncture site.

This stent can be delivered to the target location within the blood vessel via combination of guide and delivery catheters. The operator takes care to orient the stent so that the polymer region is aligned with the portion of the vessel that is targeted for puncture. The operator then deploys the stent in the vessel using a puncture system (such as described above) to puncture through the polymer region of the stent device.

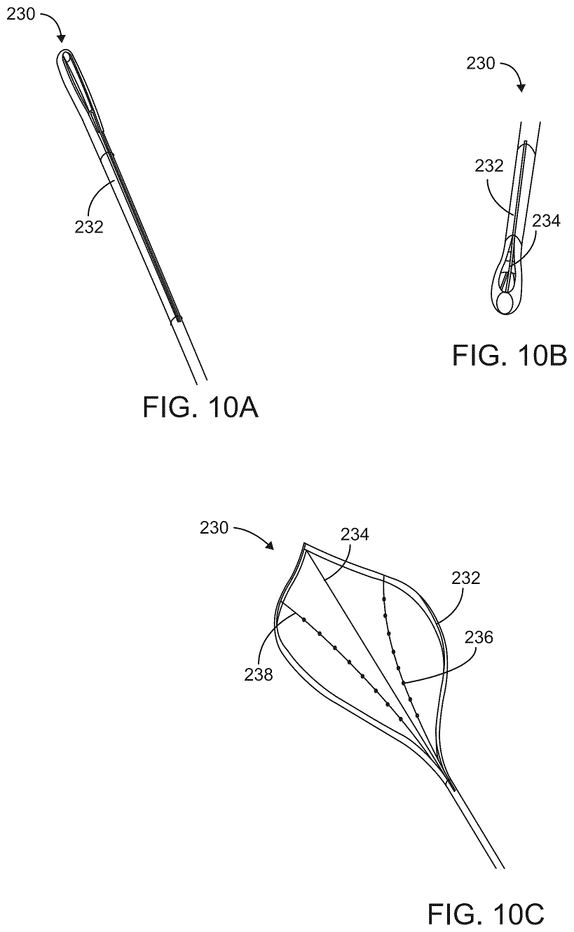

FIGS. 10A to 10C illustrate a variation of an electrode carrier 230 for deployment through the vessel wall. FIG. 10A shows a partial perspective view of the electrode carrier 230 in a delivery configuration where the electrodes are contained between adjacent arms 232. FIG. 10B shows a view of the electrode carrier 230 from a front of the carrier 230 showing a pull wire 234 located between adjacent arms 232. The arms 232 allow the electrode carrier 230 to have an atraumatic shape for navigation to the vessel site. using a catheter system.

The low cross-sectional area/crossing profile that is achieved in the collapsed state allows the device to be delivered transvascular with a mitigated risk of gross vessel damage. As shown in FIG. 10C, the pull wire 234 can retract to expand the arms 232 to expose one or more splines 238 carrying one or more electrodes 236. In some variations, the splines 238 are configured to be electrodes. The ability to expand the electrode carrier in a planar direction allows the electrode array to monitor of a larger area with any number of electrodes.

To change device state into a 2-dimensional recording/stimulation array, a proximal feature on the lead is actuated by the operator. This retracts the distal tip of the device, causing a slit section of the lead to separate and bow outwards. Several rib features that are connected to the inner surface of the lead outer jacket and nested within the collapsed lead, then splay out as the bowing occurs. Each of the rib features contains a multitude of electrodes which are spread in a 2-dimensional spatial array across the brain surface. Additional features such as small mesh pads may be incorporated into the design to promote targeted endothelialization as a method of securing the array in place.

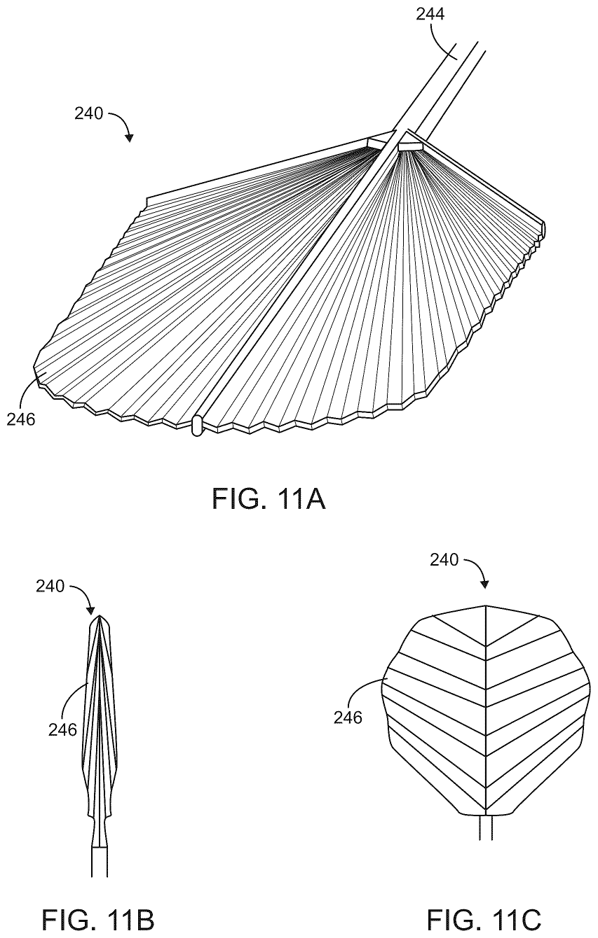

FIGS. 11A to 11C illustrate a variation of an electrode carrier 240 having a folding array 246 containing any number of electrodes where the array is located at the end of a catheter shaft 244. The that interchanges between two deployment states as shown in FIGS. 11B and 11C. The collapsed state allows the electrode carrier 240 to be delivered through catheters/vascular system to the target site), and then expanded (for end recording use). The collapsed state, shown in FIG. 11B, produces a conformal array with the minimum crossing profile of the array to allow safe passage through small vessels and catheter systems. The expanded planar state, shown in FIG. 11C, produces a flexible, 2D array with the ability to arrange recording/stimulation electrodes spatially across the surface to achieve a maximized recording area. Expansion of the array can be achieved through various methods including operator actuation using cable features, self-expanding rib/spine features, thermally actuating polymers, fluid absorption actuation polymers, etc.

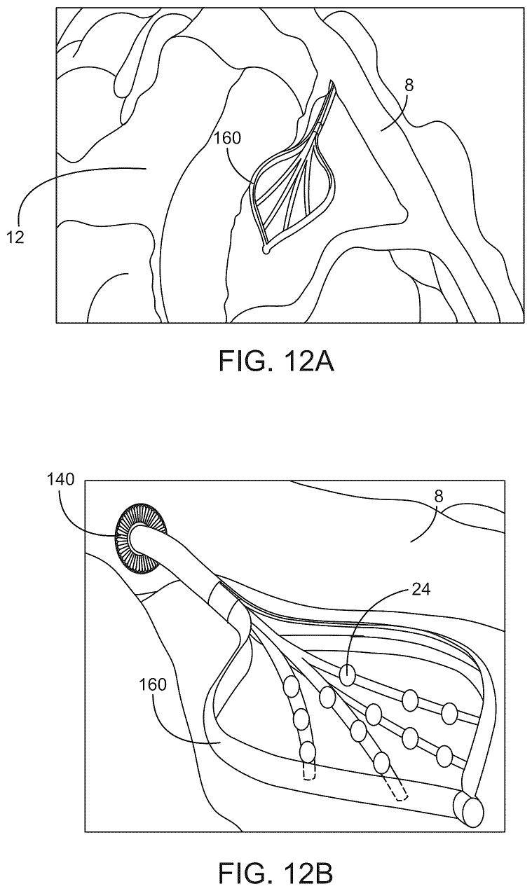

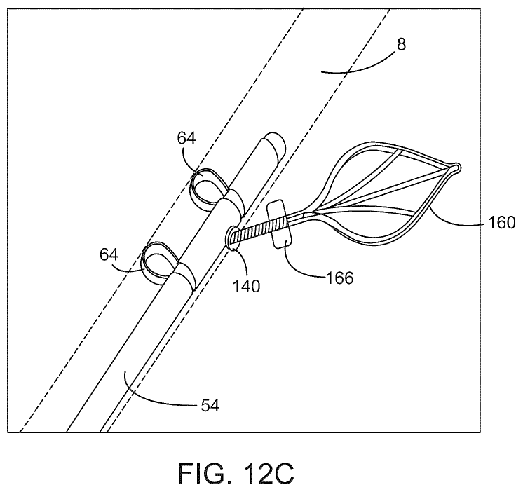

FIGS. 12A to 12C illustrate another variation of an expandable electrode carriers 160 extending out of a superior sagittal sinus 8 for positioning over brain tissue 12. FIG. 12A illustrates the electrode carrier 160 in a deployed state for recording neural signals. As noted above, the collapsed state of the carrier 160 provides a conformal array with the minimum crossing profile to permit safe passage through small vessels and other catheter systems. The expanded state shown in FIGS. 12A and 12B produce a flexible, 2D array with recording/stimulation electrodes 24 arranged spatially across the surface to achieve a maximized recording area. Expansion of the array can be achieved through various methods including operator actuation using cable features, self-expanding rib/spine features, thermally actuating polymers, fluid absorption actuation polymers, etc. As shown, the electrode carrier 160 can advance through the superior sagittal sinus (SSS) 8 or other vessels through a fixation grommet 140.

FIG. 12C illustrates a guide catheter 54 within a SSS 8, where the catheter includes expanding features 64 to secure a side port against a wall of the SSS vessel 8. In this variation, the device 160 extends through the vessel wall and through a self-expanding puncture port 140 similar to the grommets described here. In addition, the device 160 can include one or more fixation tabs 166 that permits endothelization to the dura or cortical surface to secure the electrode carrier 160 to a desired location.

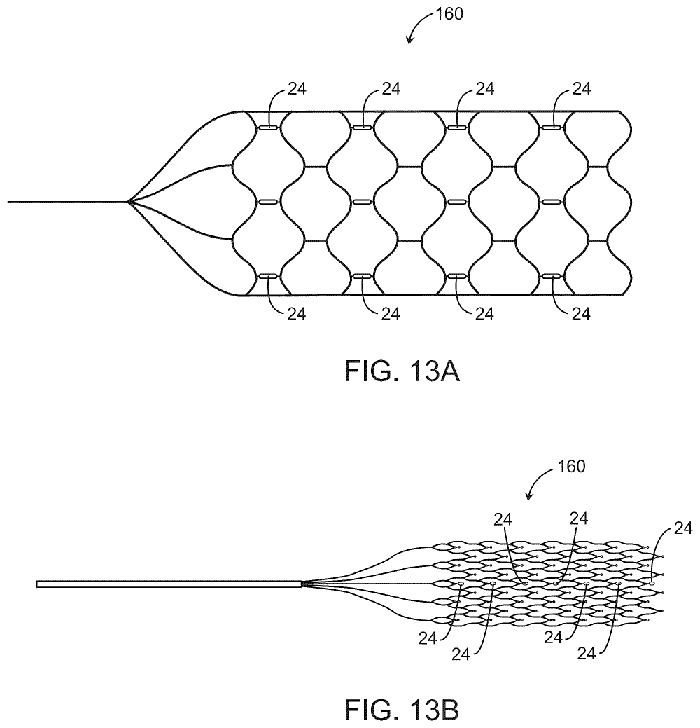

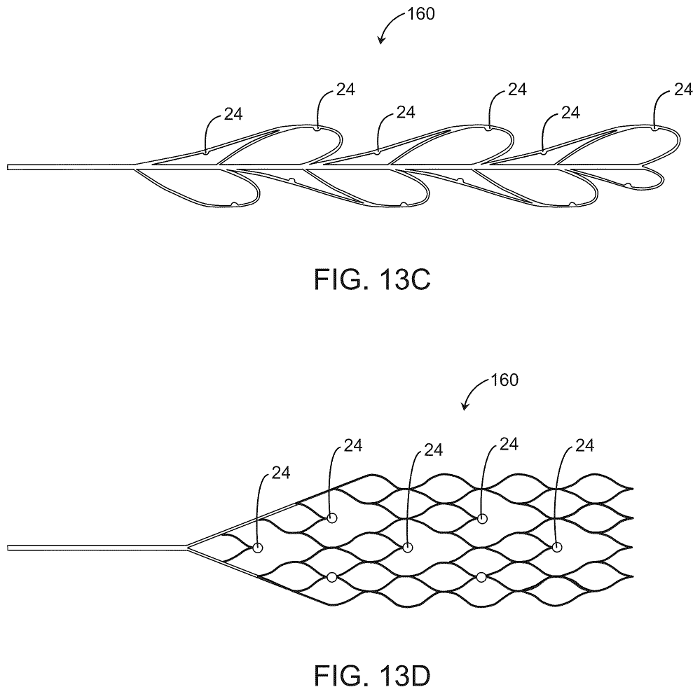

FIGS. 13A to 13D illustrate additional variations of devices 160 that carry various electrodes 24, where such devices provide stent electrode arrays that can be planar or flexible in shape. Such examples are similar to the tubular designs shown in the applications and patents referenced below. However, the devices 160 can be configured to have a flexibility allowing for the electrodes 24 to cover more brain tissue as needed. For example, such planar configurations can conform to regions of the brain (e.g., the subarachnoid space).

Additional examples of tubular structure that can be reconfigured into planar electrode carriers can be found in the following patents and provisional applications 10,575,783; 10,485,968; 10,729,530; 63/370,164; 63/517,495 and 63/370,169. The entirety of each of which is incorporated by reference.

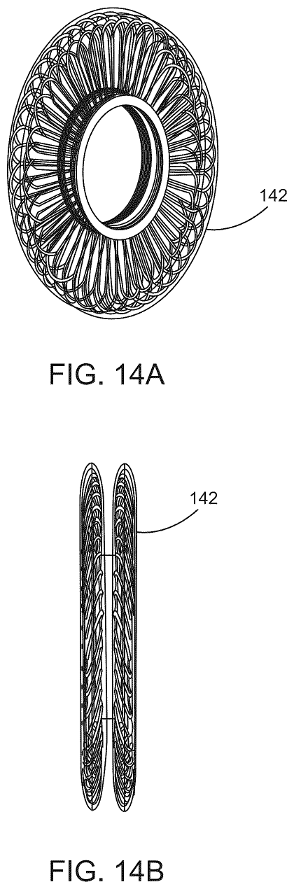

FIGS. 14A and 14B illustrate perspective and side views respectively of an example of an expanding vessel puncture port or grommet 142.

It is noted that the concepts above, while being illustrated as separate applications, can be combined in whole or in part.

All existing subject matter mentioned herein (e.g., publications, patents, patent applications) is incorporated by reference herein in its entirety except insofar as the subject matter may conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

Reference to a singular item includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation. Unless defined otherwise, all technical and scientific terms used herein have the same meaning as commonly understood by one of ordinary skill in the art to which this invention belongs.

In understanding the scope of the present disclosure, the term “comprising” and its derivatives, as used herein, are intended to be open-ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Also, the terms “part,” “section,” “portion,” “member” “element,” or “component” when used in the singular can have the dual meaning of a single part or a plurality of parts. As used herein, the following directional terms “forward, rearward, above, downward, vertical, horizontal, below, transverse, laterally, and vertically” as well as any other similar directional terms refer to those positions of a device or piece of equipment or those directions of the device or piece of equipment being translated or moved. Finally, terms of degree such as “substantially”, “about” and “approximately” as used herein mean a reasonable amount of deviation (e.g., a deviation of up to ±0.1%, ±1%, ±5%, or ±10%, as such variations are appropriate) from the specified value such that the end result is not significantly or materially changed.

This disclosure is not intended to be limited to the scope of the particular forms set forth, but is intended to cover alternatives, modifications, and equivalents of the variations or embodiments described herein. Further, the scope of the disclosure fully encompasses other variations or embodiments that may become obvious to those skilled in the art in view of this disclosure.

1. A system for accessing a target region of a brain from a vessel, the system comprising:
a catheter body having a distal region;
a navigation device slidably advanceable through the catheter body to the distal region, the navigation device comprising a distal portion that is configured to be steerable independently of the catheter body and an expandable member at the distal portion, where the expandable member is configured to anchor the distal portion exterior to the vessel;
a guidewire configured to extend through a working lumen of the navigation device; and
an electrode carrier configured to be advanced through the working lumen of the navigation device and through the expandable member such that the electrode carrier can be advance in a straight line from an opening in the expandable member to the target region of the brain.

2. The system of claim 1, further comprising a first expandable structure located at the distal region of the catheter body and configured to bias the catheter body against a wall of the vessel.

3. The system of claim 1, wherein the catheter body comprises a passage exiting a side opening in a sidewall at the distal region, wherein the passage is configured such that advancement of the navigation device therethrough causes the navigation device to exit the catheter body at the side opening.

4. The system of claim 1, further comprising a bone penetrating structure configured for sliding through the catheter body.

5. The system of claim 1, wherein the distal portion of the navigation device is steerable.

6. The system of claim 1, wherein the electrode carrier comprises a linear electrode array.

7. The system of claim 1, wherein the electrode carrier comprises a planar electrode region configured to have a delivery profile and expandable to a planar profile when advanced out of the navigation device.

8. The system of claim 7, wherein the planar electrode region comprises a foldable structure such that expansion of the planar electrode region from the delivery profile to the planar profile comprises unfolding the foldable structure.

9. The system of claim 7, wherein the planar electrode region comprises an expandable structure such that expansion of the planar electrode region from the delivery profile to the planar profile comprises expanding the expandable structure to expose one or more electrodes.

10. The system of claim 7, wherein the planar electrode region comprises a flexible electrode array.

11. The system of claim 1, further comprising a grommet structure configured for placement within an opening in a wall of the vessel, where the grommet structure allows passage of the catheter body or navigation device therethrough.

12. The system of claim 1, further comprising a stent structure having at least one opening in a side of the stent structure for passage of the catheter body or navigation device therethrough when positioned in the vessel.

13. The system of claim 12, where the at least one opening in the stent structure comprises a port.

14. The system of claim 13, wherein the port comprises a sharp edge opposite to the stent structure.

15. The system of claim 14, wherein the port is covered by a degradable or dissolvable polymer.

16. The system of claim 14, wherein the port is removable from the stent structure.

17. The system of claim 13, further comprising a polymer layer covering the at least one opening in the stent structure.

18. A stent comprising:
a stent body expandable from a deployment configuration to an expanded configuration;
a port extending from a side of the stent body, the port having a passage and having a sharp edge on a free end of the port opposite to the stent body;
a polymer covering the port and the sharp edge, wherein the polymer is configured to dissolve or degrade over a period of time, wherein when deployed in a vessel the stent body biases the polymer covering the sharp edge against a wall of the vessel, wherein after the polymer dissolves or degrades, the stent body urges the sharp edge of the port into the wall of the vessel such that the wall of the vessel adheres to a portion of the port to secure the port in place.

19. The stent of claim 18, wherein the port is removable from the stent.

20. A method of transvascular access of a region of a brain, the method comprising:
advancing a catheter into a vessel;
anchoring the catheter within the vessel;
passing the catheter through a vessel opening in a wall of the vessel and adjacent to brain tissue;
deploying a navigation device from the catheter to an exterior of the vessel;
expanding an expandable structure located at a distal portion of the catheter, where the expandable structure anchors to the exterior of the vessel;
steering the expandable structure to align a travel path from an opening of the expandable structure to a target region; and
advancing an electrode carrier from the opening of the expandable structure along the travel path and to the target region.

21. The method of claim 20, wherein advancing the electrode carrier from the opening of the expandable structure along the travel path and to the target region comprises advancing the electrode carrier over a surface of the brain.

22. The method of claim 20, wherein advancing the electrode carrier from the opening of the expandable structure along the travel path and to the target region comprises advancing the electrode carrier through a tissue of the brain.

23. The method of claim 20, further comprising deploying a grommet in the vessel opening.

24. The method of claim 20, further comprising determining an orientation of the expandable structure to determine the travel path using an external imaging device.

25. The method of claim 20, further comprising expanding the electrode carrier in a planar a planar direction over the target region.

26. The method of claim 25, wherein the electrode carrier is configured to form a two dimensional array when expanded.

27. The method of claim 25, wherein expanding the electrode carrier in the planar direction comprises unfolding the electrode carrier from a folded state.

28. The method of claim 20, further comprising expanding an expandable member on a distal portion of the catheter and proximal to the expandable structure.

29. The method of claim 20, further comprising biasing a portion of the catheter within the vessel against the wall of the vessel.

30. The method of claim 20, wherein the navigation device comprises an intermediate catheter and where the expandable structure comprises expanding ribs on the intermediate catheter.

31.-48. (canceled)
